(19) United States
(12) Patent Application Publication
(10) Pub. No.: US 2023/0018079 A1
(43) Pub. Date: Jan. 19, 2023

(54) GENOMIC SCARRING ASSAYS AND RELATED METHODS

(71) Applicant: Agilent Technologies, Inc., Santa Clara, CA (US)

(72) Inventors: Jurgen DEL FAVERO, Niel (BE); Joachim DE SCHRIJVER, Niel (BE); Charlotte DE VOGELAERE, Niel (BE); Lien HEYRMAN, Niel (BE); Dirk GOOSSENS, Niel (BE)

(21) Appl. No.: 17/785,300

(22) PCT Filed: Dec. 15, 2020
(86) PCT No.: PCT/EP2020/086255

§ 371 Date: Jun. 14, 2022

Related U.S. Application Data
(60) Provisional application No. 62/948,640, filed on Dec. 16, 2019.

(51) Int. Cl. C12Q 1/6827; G16B 20/00; G16H 50/20; G16H 50/30

(52) U.S. Cl. CPC C12Q 1/6827; G16B 20/00; G16H 50/20; G16H 50/30; C12Q 2537/143; C12Q 2537/16; C12Q 2537/165

(57) ABSTRACT

The present disclosure provides methods for detecting or predicting genomic scarring, for use in the field of diagnostic assays and for selecting treatment regimens for human diseases such as cancer.

### CROSS-REFERENCE TO RELATED APPLICATIONS

This application claims the benefit of U.S. Provisional Application Ser. No. 62/948,640, entitled “Genomic Scarring Assays” and filed on Dec. 16, 2019, which is expressly incorporated by reference herein in its entirety.

### TECHNICAL FIELD

The disclosure generally relates to methods for detecting or predicting genomic scarring, for use in the field of diagnostic assays and for selecting treatment regimens for human diseases (e.g., cancer).

### BACKGROUND

Homologous recombination (HR) is one of the primary mechanisms involved in restoring double-strand DNA breaks (DSBs). When the HR pathway is disrupted in a cell, double-stranded breaks may not be repaired efficiently (or at all), resulting in genomic instability (e.g., mutations, copy number alterations and structural rearrangements). Genomic instability resulting from HR repair deficiency, also referred to as “genomic scarring,” is in turn associated with various types of cancer. For example, copy number alterations may result in overexpression of genes due to the presence of additional copies of the gene, or low or no expression due to a loss of heterozygosity. HR repair deficiencies (HRRDs) have been observed in many types of cancer.

Poly (ADP-ribose) polymerases (PARPs) are known to play an important role in various cellular processes, including replication, recombination, chromatin remodeling, and DNA repair. Several types of tumors (e.g., BRCA1/2 mutants) have been found to have deficient HR repair pathways, and as such depend on PARP-mediated base excision repair for survival. In view of these findings, PARP inhibition has emerged as a potential strategy to selectively kill cancer cells by inactivating complementary DNA repair pathways. However, given that PARP inhibitors are generally effective only against cancers which have HR repair deficiencies, it is important to determine whether a patient has a HR repair deficient cancer prior to administration of this therapy. There are at least two commercially-available genomic scarring assays at this time, the myChoice® CDx assay offered by Myriad Genetics, Inc., and the FoundationFocus™ CDx BRCA LOH assay offered by Foundation Medicine, Inc. However, both assays require a significant amount of sequencing data as a prerequisite, e.g., the myChoice assay requires sequencing 50,000 single nucleotide polymorphism (SNP) targets and 99% of the bases must have 100 reads with the average coverage exceeding 500×, and the FoundationFocus assay requires >500× median coverage with >99% of exons at coverage >100×. This need for substantial sequencing data increases costs and processing time, limiting the usefulness of these assays.

### SUMMARY OF VARIOUS ASPECTS

In a general aspect, the disclosure provides methods for detecting or predicting homologous recombination repair deficiency (“HRRD”). In some aspects, such methods may be used to select a cancer treatment for a subject in need thereof. Such methods provide various advantages compared to known methods as described herein. For example, in some aspects, the present methods require less sequencing capacity and are consequently less expensive than known methods. Moreover, implementations of the present methods allow for detection of HRRD using standard polymerase chain reaction (PCR) equipment and are compatible with a variety of sample types (e.g., DNA extracted from fresh frozen tissue or formalin fixed paraffin embedded tissue, as well as cell-free DNA). In some aspects, the present methods may be performed using sequencing data generated by a multiplex PCR assay targeting approximately 5,000 or fewer SNPs. Such methods may be performed, e.g., as a single-tube PCR assay, saving time and resources.

In one aspect, the disclosure relates to a method for predicting HRRD, comprising the steps of: a) providing a biological specimen obtained from a human subject, wherein the specimen comprises genomic DNA; b) performing a multiplex polymerase chain reaction (PCR) assay on the genomic DNA to generate an amplified product, wherein the PCR assay is configured to amplify a plurality of amplicons; c) sequencing at least a portion of the amplified product to generate sequencing results; and d) determining a set of parameters of the biological specimen based on the sequencing results, wherein the set of parameters comprises: i) a segment size parameter, ii) a breakpoint count per unit-length parameter, and iii) a copy number parameter.

In some aspects, the method further comprises: e) predicting whether the biological specimen was obtained from a cell, tissue, or tumor that has an HRRD based upon the determined set of parameters. In some aspects, the method further comprises: selecting a treatment for the human subject from which the biological specimen was obtained based upon the determined set of parameters. In some aspects, the method further comprises: predicting the human subject's response to a cancer treatment regimen comprising a DNA damaging agent, an anthracycline, a topoisomerase I inhibitor, radiation, and/or a PARP inhibitor, based upon the determined set of parameters. In some aspects, the method may further comprise any combination of the steps described in this paragraph. In some aspects, the segment size parameter, the breakpoint count per unit-length parameter, and the copy number parameter may be aggregated (e.g., using addition or a more complex algorithm) to generate a single metric or score, prior to performing any of the predicting or selecting steps described herein.

In some aspects, the segment size parameter is determined by: identifying a plurality of segments, wherein a segment is defined as a part of the genomic DNA consisting of at least 3 consecutive amplicons containing a heterozygous polymorphic position, which have the same copy number; determining a segment size distribution for the identified plurality of segments; and calculating the posterior probability of a mixture component describing the segment size which was determined by mixture modeling on a development set. In some aspects, the segment size parameter is determined by: identifying a plurality of segments, wherein a segment is defined as a part of the genomic DNA consisting of at least 3 consecutive amplicons containing a heterozygous polymorphic position, which have the same copy number; and calculating a mean segment size for the identified plurality of segments. In some aspects, the plurality of segments comprises segments having a size within the range of 5-50 megabase pairs (MBp). In some aspects, the segments are each within the range of 1-10, 10-20, 20-30, 30-40, 40-50, 50-60, 60-70, 70-80, 80-90, or 90-100 MBp in length. In some aspects, the segments may be longer (e.g., any length up to the length of a full chromosomal arm). In some aspects, the segments are each at least 5, 10, 15, 20, 25, 30, 35, 40, 45, 50, 60, 70, 80, 90 or 100 MBp in length. In some aspects, the segments are each less than 5, 10, 15, 20, 25, 30, 35, 40, 45, 50, 60, 70, 80, 90 or 100 MBp in length.

In some aspects, the breakpoint count per unit-length parameter is determined by calculating the number of breakpoints per 5, 6, 7, 8, 9, 10, 11, 12, 13, 14, 15 Mb of the genomic DNA. In some aspects, the breakpoint count per unit-length may be calculated for a portion of the genomic DNA (e.g., it may be calculated for one or more chromosomes or chromosome arms present within the genomic DNA). In some aspects, the breakpoint count per unit-length parameter is determined by calculating the posterior probability of the mixture component describing the number of breakpoints determined by mixture modeling on a development set.

In some aspects, the copy number parameter is determined by calculating the number of copies of one or more segments of the genomic DNA, wherein a segment is defined as a part of the genomic DNA consisting of at least 3 consecutive amplicons containing a heterozygous polymorphic position, which have the same copy number. In other aspects, a segment as required by any of the methods disclosed herein may be defined as a part of the genomic DNA consisting of at least 1, 2, 3, 4, 5, 6, 7, 8, 9, 10 (or any other arbitrary number) of consecutive amplicons containing a heterozygous polymorphic position, which have the same copy number. In some aspects, the copy number parameter may be calculated based upon a plurality of segments of the genomic DNA (e.g., at least 5, 10, 15, 20, 25, 30, 35, 40, 45, 50, 100, 150, 200, 250, or 500 segments). In some aspects, the number of copies for each of the one or more segments may be selected from: near-diploid, near-tetraploid, near-hexaploid, near-octaploid, or near any other ploidy number. In some aspects, the number of copies may be represented numerically, e.g., the copy number parameter may comprise a numerical mean or median copy number for a plurality of segments of the genomic DNA. In some aspects, the copy number parameter is: a) based on a plurality of segments of the genomic DNA, and calculated by determining the posterior probability of a mixture component describing the copy number of the plurality of segments, which was determined by mixture modeling on a development set; and/or b) based at least in part on a categorization of the plurality of segments based upon their respective ploidy values.

In some aspects, the biological specimen was obtained from a human tissue, tumor, or cell. The biological specimen may be obtained from a healthy human subject or from a human subject that has been diagnosed with or is suspected of having a cancer.

In some aspects, the genomic DNA comprises a euploid genome and the PCR assay is configured to amplify at least 1,000; 2,000; 3,000; 4,000; 5,000; 6,000; 7,000; or 8,000 amplicons.

In some aspects, each amplicon contains at least one polymorphic position having an average population frequency of a minor allele of at least 0.1, 0.5, 1, 2, 3, 4, 5, 6, 7, 8, 9, 10, 11, 12, 13, 14, 15, 16, 17, 18, 19, 20, 21, 22, 23, 24, or 25%.

In another general aspect, the disclosure provides a method for detecting HRRD, comprising the steps of: a) performing a multiplex polymerase chain reaction (PCR) assay on genomic DNA obtained from a human subject, to generate an amplified product comprising a plurality of amplicons which contain single-nucleotide polymorphisms (SNPs); b) determining beta-allele frequency (BAF) and copy number parameters for each of the SNPs; c) identifying a plurality of genomic segments, based on the BAF and copy number parameters for each of the SNPs, using an ASCAT algorithm; d) determining the posterior probabilities for three components of a mixture model, using the genomic segments, wherein the components comprise a segment size, a breakpoint count per unit-length, and a copy number; and e) calculating an HRRD score using a linear model, based on the posterior probabilities for the three components of the mixture model.

In some aspects, the method further comprises step f) predicting whether the genomic DNA was obtained from a cell, tissue, or tumor that has an HRRD based upon the HRRD score. In other aspects, step f) may comprise predicting the human subject's response to a cancer treatment regimen comprising a DNA damaging agent, an anthracycline, a topoisomerase I inhibitor, radiation, and/or a poly ADP-ribose polymerase (PARP) inhibitor, based upon the HRRD score.

In some aspects, the segment size component is determined by: identifying a plurality of segments, wherein a segment is defined as a part of the genomic DNA consisting of at least 3 consecutive amplicons containing a heterozygous polymorphic position, which have the same copy number; determining a segment size distribution for the identified plurality of segments; and calculating the posterior probability of a mixture component describing the segment size which was determined by mixture modeling on a development set.

In some aspects, the plurality of segments comprises segments which each have: a) a size within the range of 5-50 megabase pairs (MBp); b) a size within the range of 1-10, 10-20, 20-30, 30-40, or 40-50 MBp in length; c) a size of at least 5, 10, 15, 20, 25, 30, 35, 40, 45, or 50 MBp

In some aspects, the genomic DNA was extracted from a biological specimen a) taken from a healthy human subject orb) taken from a human subject that has been diagnosed with or is suspected of having a cancer. The genomic DNA may comprise, e.g., a euploid genome and the PCR assay is configured to amplify at least 1,000; 2,000; 3,000; 4,000; 5,000; 6,000; 7,000; or 8,000 amplicons. In some aspects, each amplicon contains at least one polymorphic position having an average population frequency of a minor allele of at least 10, 11, 12, 13, 14, 15, 16, 17, 18, 19, 20, 21, 22, 23, 24, or 25%.

In another general aspect, the disclosure provides a method for predicting HRRD, comprising: a) providing an electronic device comprising one or more processors; b) receiving, by the electronic device, sequencing results for an amplification product generated by a multiplex PCR using genomic DNA obtained from a human subject, wherein the sequencing results comprise sequences for a plurality of amplicons which contain SNPs; c) determining, by the electronic device, beta-allele frequency (BAF) and copy number parameters for each of the SNPs; d) identifying, by the electronic device, a plurality of genomic segments, based on the BAF and copy number parameters for each of the SNPs, using an ASCAT algorithm; e) determining, by the electronic device, the posterior probabilities for three components of a mixture model, based on the genomic segments, wherein the components comprise a segment size, a breakpoint count per unit-length, and a copy number; and f) calculating, by the electronic device, an HRRD score using a linear model, based on the posterior probabilities for the three components of the mixture model.

In another general aspect, the disclosure provides a system for predicting HRRD, comprising: an electronic device comprising one or more processors, configured to receive sequencing results for an amplification product generated by a multiplex PCR using genomic DNA obtained from a human subject, wherein the sequencing results comprise sequences for a plurality of amplicons which contain SNPs; determine beta-allele frequency (BAF) and copy number parameters for each of the SNPs; identify a plurality of genomic segments, based on the BAF and copy number parameters for each of the SNPs, using an ASCAT algorithm; determine the posterior probabilities for three components of a mixture model, based on the genomic segments, wherein the components comprise a segment size, a breakpoint count per unit-length, and a copy number; and calculate an HRRD score using a linear model, based on the posterior probabilities for the three components of the mixture model. In some aspects, the disclosure provides a an electronic device comprising one or more processors, configured to perform one or more steps of any of the methods described herein.

In another general aspect, the disclosure provides methods of amplifying genomic DNA, comprising: a) obtaining genomic DNA from a specimen obtained from a human subject known to or suspected of having a cancer; and b) amplifying a plurality of amplicons which contain single-SNPs by performing a multiplex PCR using the genomic DNA; wherein the multiplex PCR is performed using a set of PCR primers configured to amplify at least 5,000 amplicons spanning across all 22 human somatic chromosomes, wherein each amplicon comprises a SNP. In some aspects, each amplicon comprises a maximum length of 100 bp. In some aspects, the average amplicon density is 1 amplicon per 400-600 kb of somatic chromosome DNA. In some aspects, the plurality of amplicons includes one or more portions of each of the following genes: BRCA1, BRCA2, BRIP1, RAD51C, RAD51D, ATM, BARD1, CHEK1, CHEK2, FANCA, FANCL, NBN, PALB2, RAD51B, RAD54L, CDK12, and TP53.

In another general aspect, the disclosure provides methods of generating a PCR amplification product, comprising: a) obtaining genomic DNA from a specimen obtained from a human subject (e.g., known to have or suspected of having a cancer); and b) generating the PCR amplification product by amplifying a plurality of amplicons which each contain a single-nucleotide polymorphisms (SNP) by performing a multiplex PCR using the genomic DNA; wherein the multiplex PCR is performed using a set of PCR primers configured to amplify at least 5,000 amplicons spanning across all 22 human somatic chromosomes. In some aspects, each amplicon comprises a maximum length of 100 bp. In some aspects, the average amplicon density is 1 amplicon per 400-600 kb of somatic chromosome DNA. In some aspects, the plurality of amplicons includes one or more portions of each of the following genes: BRCA1, BRCA2, BRIP1, RAD51C, RAD51D, ATM, BARD1, CHEK1, CHEK2, FANCA, FANCL, NBN, PALB2, RAD51B, RAD54L, CDK12, and TP53.

In another general aspect, the disclosure provides methods of treating a cancer, comprising: a) receiving, by an electronic device, sequencing results for an amplification product generated by a multiplex PCR using genomic DNA obtained from a tumor found in a human subject, wherein the sequencing results comprise sequences for a plurality of amplicons which contain SNPs; b) determining, by the electronic device, BAF and copy number parameters for each of the SNPs; c) identifying, by the electronic device, a plurality of genomic segments, based on the BAF and copy number parameters for each of the SNPs, using an ASCAT algorithm; d) determining, by the electronic device, the posterior probabilities for three components of a mixture model, based on the genomic segments, wherein the components comprise a segment size, a breakpoint count per unit-length, and a copy number; and e) calculating, by the electronic device, an HRRD score using a linear model, based on the posterior probabilities for the three components of the mixture model; and f) selecting and/or administering a cancer treatment for the subject based on the HRRD. In other aspects, the disclosure provides a method of treating a cancer based on any of predictive methods described herein.

In some aspects, the cancer treatment is administration of a DNA damaging agent, an anthracycline, a topoisomerase I inhibitor, radiation, and/or a PARP inhibitor. In some aspects, the selected cancer treatment is administration of a PARP inhibitor when the HRRD score is above (or below) a preselected threshold.

In some aspects, a method of treating a cancer according to the disclosure comprises: a) obtaining genomic DNA from a specimen obtained from a human subject known to or suspected of having a cancer; and b) amplifying a plurality of amplicons which contain single-nucleotide polymorphisms (SNPs) by performing a multiplex PCR using the genomic DNA, wherein the multiplex PCR is performed using a set of PCR primers configured to amplify at least 5,000 amplicons spanning across all 22 human somatic chromosomes, wherein each amplicon comprises a SNP; c) determining an HRRD score for the human subject, based on the sequences of the plurality of amplicons; and d) selecting and/or administering a cancer treatment for the subject based on the HRRD. In some aspects, the cancer is an ovarian cancer (e.g., a platinum-sensitive ovarian cancer or a platinum-resistant ovarian cancer.

In still further aspects, the disclosure provides kits for amplifying genomic DNA using a multiplex PCR assay, wherein the kit comprises a) a PCR reaction mixture; b) a DNA polymerase; and c) a set of PCR primers, wherein the set of PCR primers is configured to amplify at least 5,000 amplicons spanning across all 22 human somatic chromosomes, wherein each amplicon comprises a SNP and a maximum length of 100 bp.

In some aspects, the average amplicon density is 1 amplicon per 400-600 kb, 300-700 kb, or 500-560 kb of somatic chromosome DNA. In some aspects, the amplicons amplify one or more portions of each of the following genes: BRCA1, BRCA2, BRIP1, RAD51C, RAD51D, ATM, BARD1, CHEK1, CHEK2, FANCA, FANCL, NBN, PALB2, RAD51B, RAD54L, CDK12, and TP53.

Additional aspects will be readily apparent to one of skill in light of the totality of the disclosure.

### BRIEF DESCRIPTION OF THE DRAWINGS

FIG. 1 is a flowchart showing an exemplary method for predicting HRRD according to the disclosure.

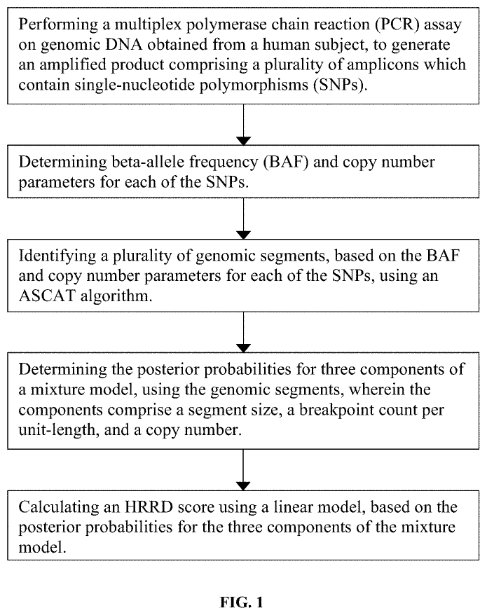

FIG. 2 is a diagram showing an exemplary set of amplicon positions that may be used with the methods described herein, mapped across the 22 autosomal chromosomes of the human genome.

FIG. 3 is a diagram showing an exemplary set of samples sorted based upon their genomic scarring score with mutational information provided. The genomic scarring score increases from left to right along the x-axis.

FIG. 4 shows a set of graphs comparing the copy number (normalized values, log R) and beta allele frequency (BAF) and for samples analyzed with a genomic scarring assay according to the present methods versus with a SNP array.

FIG. 5 shows a set of three graphs that compare predicted HRRD scores generated using the present methods against known metrics for genomic scarring determined using a SNP array.

FIG. 6 is a graph showing HRRD scores in relation to homologous recombination repair pathway gene mutation status and best confirmed response to Olaparib monotherapy in a cohort of patients with relapsed ovarian cancer.

FIG. 7 illustrates an example of a general-purpose computer system on which the disclosed systems and methods may be implemented.

### DETAILED DESCRIPTION OF VARIOUS ASPECTS

The present disclosure provides various methods for determining or predicting HRRD. In some aspects, the method may comprise the steps of: a) providing a biological specimen obtained from a human subject, wherein the specimen comprises genomic DNA; b) performing a multiplex PCR assay on the genomic DNA to generate an amplified product, wherein the PCR assay is configured to amplify a plurality of amplicons; c) sequencing at least a portion of the amplified product to generate sequencing results; and d) determining a set of parameters of the biological specimen based on the sequencing results. In some aspects, the set of parameters comprises: i) a segment size parameter, ii) a breakpoint count per unit-length parameter, and iii) a copy number parameter. In other aspects, the method comprises performing a multiplex PCR assay on genomic DNA from a biological specimen obtained from a human subject to generate an amplified product, wherein the PCR assay is configured to amplify a plurality of amplicons; sequencing at least a portion of the amplified product to generate sequencing results; and determining a set of parameters of the biological specimen based on the sequencing results. The present methods provide multiple advantages compared to known genomic scarring assays, including reducing cost and processing time (e.g., due to lower sequencing requirements), and by providing a clinically useful diagnostic for HRRD that can be performed using standard PCR equipment.

In some aspects, the set of parameters comprises: i) a segment size parameter, ii) a breakpoint count per unit-length parameter, and iii) a copy number parameter. In some aspects, these three parameters may be aggregated to generate a single score (e.g., representative of the level of genomic scarring), by addition or using a more complex algorithm. In either case, the individual parameters or an aggregate parameter based on the individual parameters, may be used as a diagnostic to predict responsiveness of the human subject to treatment with an anticancer therapy or to select an anticancer treatment.

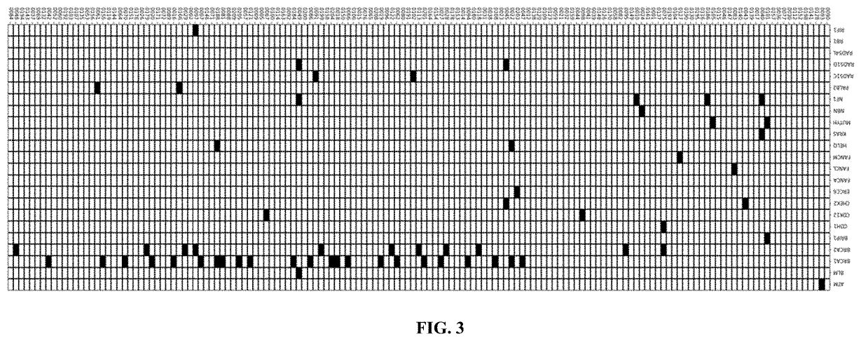

In some aspects, methods according to the disclosure may be based on a multiplex PCR assay which includes amplicons spread across a plurality of the 22 pairs of human autosomal chromosomes. An exemplary set of amplicons is shown mapped to the human genome in FIG. 3. The amplicons may target specific genes or chromosomal regions in some aspects. In other aspects, the amplicons may be spread randomly across the 22 autosomal chromosomes. In some aspects, the assay may include one or more amplicons capable of amplifying DNA on the human sex chromosomes, as well. In some aspects, the size of the amplicons may span from 70-90 base pairs (bp). However, the size of the amplicon may be varied as needed for a given implementation. For example, the amplicon size may be 1-50 bp, 50-100 bp, 100-150 bp, 150-200 bp, 200-250 bp, 250-300 bp, 300-350 bp, 350-400 bp, 400-450 bp, 450-500 bp, or >500 bp. In some aspects, the amplicon size may be within a range formed by selecting a minimum and maximum size from any of the aforementioned values. In some aspects, some or all of the selected amplicons will contain at least one polymorphic position. In some aspects, amplicon resolution may be at least or approximately 100, 150, 200, 250, 300, 350, 400, 450, 500, 550, 600, 650, 700, 750, or 800 kb. In some aspects, the primers used for the multiplex PCR assay may be selected for compatibility in a single-tube PCR reaction. The resulting PCR product may be sequenced using any devices known in the art, such as by an Illumina® sequencing instrument (e.g., an Illumina® NextSeq system).

The sequenced output must be analyzed to determine the level of homologous recombination (HR) deficiency. In some aspects, the analysis process may consist of four steps: 1) data pre-processing, 2) coverage normalization and bias correction, 3) segmentation, and 4) determination of the HRRD score. However, it is understood that this organization is merely a non-limiting example. In other aspects, the analysis process may omit any of these steps, add additional steps, and/or combine one or more of these four steps.

The data pre-processing step may comprise generating a coverage file (containing raw read counts) and a SNP file (containing B-allele frequency, “BAF” per amplicon), based upon the initial sequencing results (e.g., one or more FastQ files).

The coverage normalization and bias correction step may comprise a) determining normalized read counts (dosage quotient, “DQ”) values for each amplicon, and then (if desired) b) generating corrected DQ values which account for sequencing biases related to the type of sample. For example, formalin-fixed paraffin-embedded samples may display a sequencing length bias correctable by known algorithms.

The segmentation step may comprise determining copy number segments using the BAF values and the DQ (or corrected DQ) values generated during the coverage normalization and bias correction step. Segmentation may be calculated using the ASCAT algorithm described in Van Loo et al. “Allele-specific copy number analysis of tumors.” PNAS 107.39 (2010): 16910-16915, which is incorporated in its entirety by reference herein. For example, a multiplex PCR amplification product produced using one of the kits described herein may be sequenced and analyzed to determine the beta allele frequency (BAF) and copy number parameters for each of the SNPs or amplicons. The BAF and copy number parameters may be used as inputs for the ASCAT algorithm to identify genomic segments. In some aspects of the methods described herein, a segment is defined as a part of the genomic DNA consisting of at least 3 consecutive amplicons containing a heterozygous polymorphic position, which have the same copy number. In other aspects, the definition of a segment may be based on different number of consecutive amplicons, e.g., at least 1, 2, 4, 5, 6, 7, 8, 9, 10 (or any other arbitrary number). The size of the copy number segments may, e.g., span from approximately 5-50 MBp.

The determination of the HRRD score may comprise determining a single integer score for a given biological sample based on the segments identified in the segmentation step. This process may begin by determining the underlying distributions of several parameters of the copy number segments, including the 1) segment size, 2) breakpoint count per unit-length (e.g., 10 MBp), and 3) copy number, by mixture modeling. These underlying distributions are called components. The sum of posteriors matrix (samples×components) may be compiled, with the dimensions then reduced using non-negative matrix factorization, allowing one to generate values for each of these parameters, which provide information regarding HRRD.

The value of these three parameters may be aggregated to form a single score indicative of the level of HR repair deficiency, which may in turn be used as a clinical diagnostic to predict responsiveness or to select a treatment regimen. FIG. 3 illustrates an exemplary set of biological samples assayed using a method according to the disclosure, ranked in order according to their HRRD score. In some aspects, this HRRD score may be used to determine whether a PARP inhibitor should be administered to a human subject who has or is suspected of having cancer, to predict responsiveness to an anticancer therapeutic, or for other purposes as described herein.

In some aspects, a genomic scarring assay according to the disclosure may begin with a multiplex PCR assay on genomic DNA obtained from a human subject, to generate an amplified product comprising a plurality of amplicons that contain SNPs, with the amplification product subsequently being sequenced and analyzed to determine an HRRD score. The sequencing results may be analyzed to determine beta-allele frequency (BAF) and copy number parameters for each of the SNPs. These BAF and copy number parameters may in turn be used to partition the genomic DNA into a plurality of genomic segments using an ASCAT algorithm as described by Loo et al., as described above. Copy number feature distributions may then be derived for one or more signatures associated with these genomic segments, such as the segment size, breakpoint count per unit-length (e.g., per 10 mb), or copy number. The analysis and calculation of any of these copy number features may be performed using the methods described in Macintyre, et al. “Copy number signatures and mutational processes in ovarian carcinoma,” Nature Genetics 50.9 (2018): 1262-1270 (herein, “Macintyre”), the entire contents of which is incorporated herein by reference. For example, mixture modeling may be used to determine the underlying distribution of each component, as described e.g., in Macintyre. The sum of posteriors matrix (samples×components) may then be compiled, with the dimensions then reduced using non-negative matrix factorization, allowing one to generate values for each of these components. The resulting values can be fit to a generalized linear model (e.g., generated based on HRRD scores for a population having known clinical outcomes), in order to arrive at an aggregate HRRD score for a given specimen.

Generalized linear models may be constructed as described in Macintyre or otherwise known in the art. Users may select a desired development set as needed for a given application (e.g., based upon cancer type/stage or patient demographic factors). For example, a generalized linear model for a genomic scarring assay according to the disclosure may be constructed using a development set of ˜250 ovarian cancer samples obtained from patients with known clinical outcomes. This particular development set produced a generalized linear model having the formula: HRRD Score=a+(b×segment size)+(c×breakpoint count per unit-length (10 mb))+(d×copy number), where “a”=77465.2750; “b”=−1.1178; “c”=−515.7440; and “d”=−1.8780. Values for each of these components may be calculated for a given sample as described above and plugged-in to this generalized linear formula to calculate an HRRD score for the sample. As described in further detail in the examples provided below, HRRD scores may be calculated for a set of samples obtained from subject having known clinical outcomes in order to determine an appropriate cut-off value for classifying a sample as HRRD-positive or HRRD-negative. It is understood that this cut-off may be empirically determined as needed for a given type of cancer.

In other aspects, the disclosure provides a method of treating cancer comprising administering a cancer treatment to a human subject who has been diagnosed with an HRRD based on the methods described herein. In particular aspects, the method further comprises administering a DNA damaging agent, an anthracycline, a topoisomerase I inhibitor, radiation, and/or a poly ADP-ribose polymerase (PARP) inhibitor.

Additional methods of treating a cancer according to the disclosure may comprise: a) receiving, by an electronic device, sequencing results for an amplification product generated by a multiplex PCR using genomic DNA obtained from a tumor found in a human subject, wherein the sequencing results comprise sequences for a plurality of amplicons which contain SNPs; b) determining, by the electronic device, BAF and copy number parameters for each of the SNPs; c) identifying, by the electronic device, a plurality of genomic segments, based on the BAF and copy number parameters for each of the SNPs, using an ASCAT algorithm; d) determining, by the electronic device, the posterior probabilities for three components of a mixture model, based on the genomic segments, wherein the components comprise a segment size, a breakpoint count per unit-length, and a copy number; e) calculating, by the electronic device, an HRRD score using a linear model, based on the posterior probabilities for the three components of the mixture model; and f) selecting and/or administering a cancer treatment for the subject based on the HRRD score.

Other methods of treating a cancer according to the disclosure may comprise a) obtaining genomic DNA from a specimen obtained from a human subject known to or suspected of having a cancer; and b) amplifying a plurality of amplicons which contain SNPs by performing a multiplex PCR using the genomic DNA, wherein the multiplex PCR is performed using a set of PCR primers configured to amplify at least 5,000 amplicons spanning across all 22 human somatic chromosomes, wherein each amplicon comprises a SNP; c) determining an HRRD score for the human subject, based on the sequences of the plurality of amplicons; and d) selecting and/or administering a cancer treatment for the subject based on the HRRD score.

In some aspects, the cancer treatment may comprise administration of a DNA damaging agent, an anthracycline, a topoisomerase I inhibitor, radiation, and/or a PARP inhibitor such as Olaparib. As described herein, a user may select an HRRD score cut-off threshold for classifying whether a sample (e.g., of a tumor) is HRRD-positive or HRRD-negative. This threshold may be based on HRRD score profiles for samples (e.g., of tumors) obtained from subjects for which a known clinical outcome is available. As such, in some aspects the selected cancer treatment is administration of a PARP inhibitor when the HRRD score is above or below a preselected threshold. The cancer may be an ovarian cancer (e.g., PSOC or PROC), or any other cancer for which an HRRD scores correlate with a clinical outcome. It is understood that one may determine whether HRRD scores are useful as a diagnostic for clinical outcomes and/or treatment effectiveness by analyzing HRRD scores obtained from patients for which known clinical outcomes are available.

In another general aspect, the disclosure provides a system for predicting HRRD, comprising: an electronic device comprising one or more processors, configured to receive sequencing results for an amplification product generated by a multiplex PCR using genomic DNA obtained from a human subject, wherein the sequencing results comprise sequences for a plurality of amplicons which contain SNPs; determine beta-allele frequency (BAF) and copy number parameters for each of the SNPs; identify a plurality of genomic segments, based on the BAF and copy number parameters for each of the SNPs, using an ASCAT algorithm; determine the posterior probabilities for three components of a mixture model, based on the genomic segments, wherein the components comprise a segment size, a breakpoint count per unit-length, and a copy number; and calculate an HRRD score using a linear model, based on the posterior probabilities for the three components of the mixture model. The electronic device may be a computer (e.g., a desktop personal computer or a cloud-based server having one or more processors configured to execute instructions for carrying out any of the methods (or steps thereof) described herein. For example, such systems may be configured to receive sequencing results for a multiplex PCR amplification product, and to perform all of the downstream analyses required to calculate a HRRD score as described herein. In some aspects, the software may be configured to allow a user to select or modify a generalized linear model used to calculate the HRRD score (e.g., by selecting a score associated with a given type of cancer).

In another general aspect, the disclosure provides methods of amplifying genomic DNA or generating amplification products (e.g., using the kits described herein). For example, a method of amplifying genomic DNA may comprise: a) obtaining genomic DNA from a specimen obtained from a human subject (e.g., known to have or suspected of having a cancer); and b) amplifying a plurality of amplicons which contain SNPs by performing a multiplex PCR using the genomic DNA; wherein the multiplex PCR is performed using a set of PCR primers configured to amplify at least 5,000 amplicons spanning across all 22 human somatic chromosomes, wherein each amplicon comprises a SNP.

A method of generating a PCR amplification product may similarly comprise: a) obtaining genomic DNA from a specimen obtained from a human subject (e.g., known to have or suspected of having a cancer); and b) generating the PCR amplification product by amplifying a plurality of amplicons which each contain a single-nucleotide polymorphisms (SNP) by performing a multiplex PCR using the genomic DNA; wherein the multiplex PCR is performed using a set of PCR primers configured to amplify at least 5,000 amplicons spanning across all 22 human somatic chromosomes.

In some aspects of such methods, each amplicon may comprise a maximum length of 100-300 bp (e.g., 100 bp) and/or the average amplicon density may be 1 amplicon per 400-600 kb of somatic chromosome DNA. The plurality of amplicons may also include one or more portions of genes associated with the HR repair pathway, e.g., BRCA1, BRCA2, BRIP1, RAD51C, RAD51D, ATM, BARD1, CHEK1, CHEK2, FANCA, FANCL, NBN, PALB2, RAD51B, RAD54L, CDK12, and/or TP53.

The development, validation, and use of an exemplary genomic scarring assay according to the disclosure shall be illustrated by the following examples.

### Example 1: Development of a Genomic Scarring Assay According to the Disclosure

In some aspects, the disclosure provides a genome-wide, Multiplex PCR-based scarring assay which utilizes an approach that takes into account loss of heterozygosity (LOH) and copy number signatures. Such assays may advantageously be designed as a generic single-plex MASTR-based test for the detection of genomic scars. MASTR (Multiplex Amplification of Specific Targets for Resequencing) assays enable multiplex PCR amplification of all required coding sequences of the genes of interest in a limited number of PCR reactions. Further downstream pooling of DNA amplicons and barcoding individual samples of the MASTR assays with contemporary Next-Generation Sequencing (NGS) technologies, allows simple, high throughput and cost-effective sequencing for both research and diagnostic purposes.

The development process for this exemplary genomic scarring began with the selection of primers for multiplex PCR amplification spread across all human autosomal chromosomes with a resolution of approximately 500 kb, which corresponds to approximately 6,000 amplicons. Each amplicon was further required to contain a SNP and a maximal amplicon length of 100 bp (for cfDNA compatibility). Ideally, all primers should be compatible to allow single-plex amplification.

To that end, in silico techniques were used to select a set of primers for the this exemplary genomic scarring assay. Hereto, repetitive regions (e.g., duplicated regions, repeat masked regions and simple repeats as defined in in the UCSC Genome Browser) were masked in the human genome. Then, to force amplicons to be designed around a prevalent SNP (to allow both tumor tissue fraction and LOH to be calculated), SNPs outside the masked regions were extracted from the 1,000 Genomes database in a stepped approach. SNP prevalence was determined based on global population prevalence. After selection of the applicable SNPs the DNA sequence around this SNP was extracted from the human reference genome and putative PCR primers, allowing amplification of an amplicon containing the SNP (“SNP amplicon”), were designed.

In total, 3 batches of SNP amplicons and accompanying putative primers were determined, comprising approximately 2,000,000 SNP amplicons. Next, an in silico single-plex design was performed using a modified PCR multiplexing algorithm which was initiated with the selection of one initial highly-prevalent SNP as the first amplicon of the single-plex and each of the 2,000,000 putative SNP amplicons were added sequentially with decreasing SNP frequency prevalence and checked for compatibility with the amplicons already present in the in silico single-plex. Since computationally multiplexing a large single-plex PCR reaction is a highly non-linear process in time space because the chance of finding a compatible primer pair decreases over process time resulting in longer processing times per primer pair added. Altogether, approximately 100 computing days were required to do the effective multiplexing of the 2,000,000 putative sequences computationally into a single-tube PCR reaction. In other to speed up development of this genomic scarring assay, work to verify and optimize the computational multiplexing results proceeded in parallel while this in silico multiplexing simulation was performed.

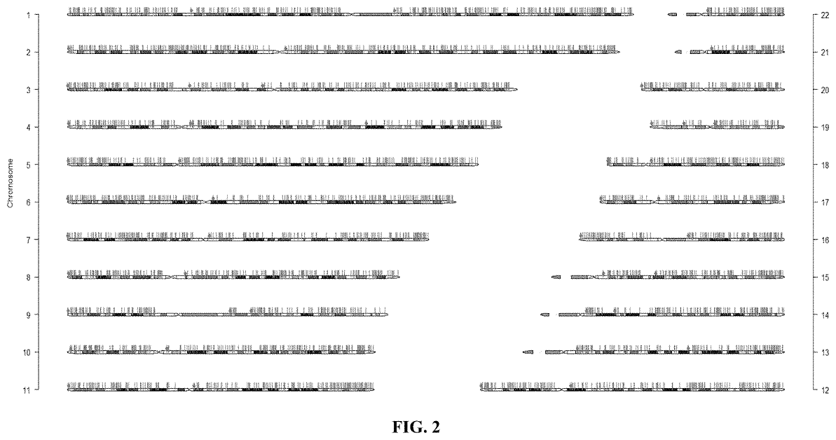

After in silico multiplexing of the first batch of approximately 300,000 SNP amplicons, 3,421 primer pairs were ordered and equimolarly mixed together (primer mix) followed by PCR amplification and NGS. Initial PCR amplification conditions (buffer composition and cycling conditions) were identical to the conditions used for Clarigo. NGS based analysis allowed to determine which primers resulted in primer dimer (PD) formation that were subsequently removed from the primer mix. This process was repeated by adding, 1,852 primer pairs from the second in silico design batch to the PD resolved primer mix and were again amplified, sequenced and analyzed for PD forming primers. This was performed a third time with primers designed to fill the gaps in the previous two batches. This third primer batch contained 756 primers and was added to the PD resolved primer mix from the first and second design. The completed primer mix was again sequenced and analyzed for PD forming primers. Next, all PD forming primers from the final primer mix were removed, the complete primer mix was remade, sequenced and analyzed for primer pairs that resulted in under- or over representation of amplicons and that showed significant amplification bias of heterozygous SNPs. Concretely, amplicons with a coverage above 5× mean coverage, amplicons with a normalized coverage below 50× the mean coverage and amplicons with an average heterozygous allele frequency outside the 40-60% range were excluded and physically removed from the assay. This resulted in a final genomic scarring assay containing 5,201 amplicons divided over all autosomal chromosomes with an average density of 1 amplicon per 531 kb. FIG. 2 illustrates the distribution of the SNP amplicons on autosomal chromosomes. In total, 6,029 primer pairs were designed in silico and ordered, of which 5,201 (86%) were retained in the final genomic scarring MASTR assay.

### Example 2: Validation of a Genomic Scarring Assay According to the Disclosure

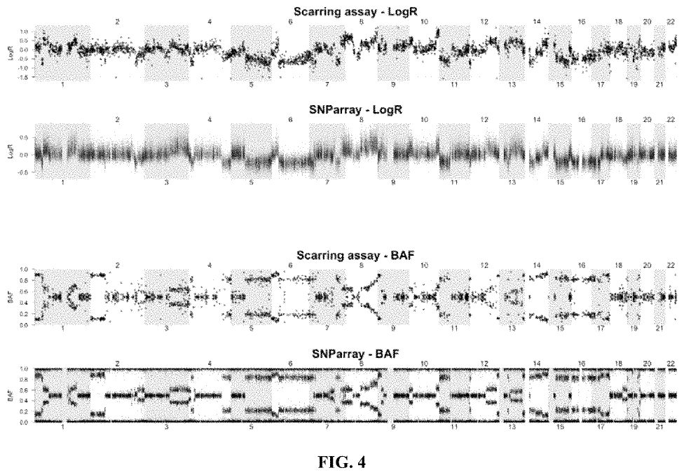

The performance of the genomic scarring assay developed in Example 1 was validated by testing the assay using reference samples that were previously analyzed with SNP arrays. A SNP array is a microarray containing immobilized allele-specific oligonucleotide probes. As illustrated by FIG. 4, the pattern of beta allele frequency (BAF) and copy number parameters (normalized using log ratio values) for each SNP was highly similar to the SNP array derived pattern, despite the fact that the data point density is 160-fold less for the genomic scarring assay.

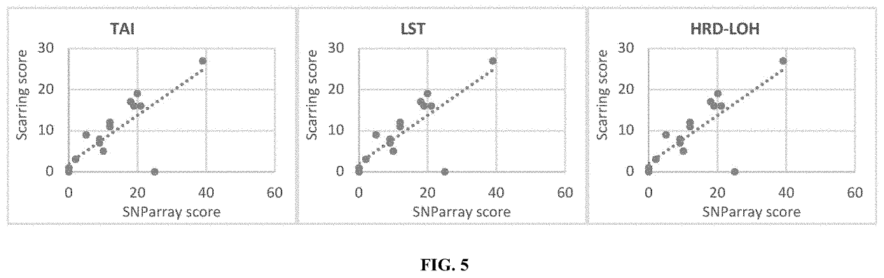

The genomic scarring assay was also tested for compatibility with a set of FFT (fresh frozen tissue), FFPE (formalin-fixed paraffin-embedded) and cfDNA (cell free DNA) samples. First, a 62 HGSOC FFT sample set was amplified using the genomic scarring assay. The amplification product was sequenced using NGS. For all these samples, TAI, LST and HRRD-LOH score were available as reference data. Scarring scores were calculated as described above, correlated to the scores calculated based on SNP array data. FIG. 5 shows the correlation for each of the scores which can be classified as good.

Second, 55 high-grade serous ovarian cancer (HGSOC) FFPE samples were analyzed with the genomic scarring assay, of which 39 samples were matched with FFT samples from the first study. This data that confirmed that genomic scarring scores could be successfully calculated for FFPE-derived DNA samples.

Third, 50 cfDNA samples from a CLIO study (NCT02822157) and matching HGSOC FFT samples from relapse tumor biopsies were analyzed using the genomic scarring assay. FFPE tissue from the primary tumor was available for each patient. Of the 154 included patients, 103 were randomized in the Olaparib cohort (and received this PARP inhibitor) and 51 were in the chemotherapy cohort. Of the latter cohort, 32 patients underwent crossover to Olaparib, which means that Olaparib response data (overall objective response, primary endpoint) was available for 135 patients. For all included patients, plasma samples, obtained prior to therapy and monthly during the trial, were available. Of this cohort of 135 patients with Olaparib response data, 105 pre-treatment cfDNA samples, 105 matched germline DNA samples and 50 matched FFT DNA samples derived from a pre-treatment biopsy were extracted. This is a highly unique clinical dataset and one of the largest in its kind. Our results demonstrated a good correlation in scarring score between the cfDNA sample and the matching FFT tumor sample, provided that a minimal tumor tissue content of 20% was present.

### Example 3: Use of the Genomic Scarring Assay to Predict PARP Inhibitor Responsiveness in Patients with Platinum-Resistant or Platinum-Sensitive Ovarian Cancer

CLIO trial NCT 02822154 evaluated Olaparib monotherapy (a PARP inhibitor) versus chemotherapy in a set of randomized patients with relapsed ovarian cancer. PARP inhibitor treatment is approved as maintenance for responding platinum-sensitive relapsed ovarian cancer (PSOC). In this study, patients with PSOC were randomly assigned to one of two initial cohorts and treated with either Olaparib or chemotherapy. In parallel, a second set of patients with platinum-resistant relapsed ovarian cancer (PROC) were randomly assigned to Olaparib or chemotherapy treatment cohorts. Subsets of both of the chemotherapy cohorts were later selected for Olaparib treatment; this cross-over design provided additional insight regarding the combination of chemotherapy and Olaparib treatment.

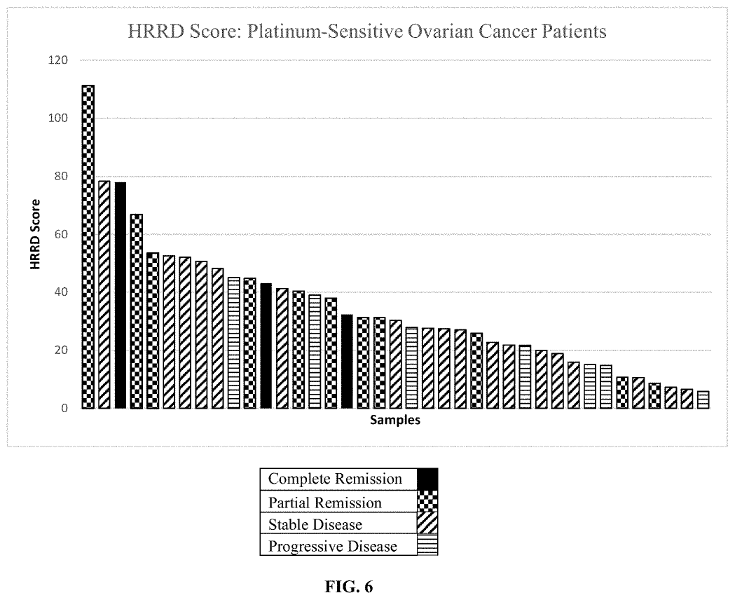

FFPE-derived primary tumor DNA was obtained from a majority of the patients and used to generate an HRRD score for each patient using a genomic scarring assay according to the disclosure. In this case, the posterior probability of the segment size, breakpoint count per unit-length, and copy number components was determined using a mixture model generated based on the analysis of 207 HGSOC cases from the UZ Leuven tumor bank. The HRRD scores were compared with patient outcomes observed upon a follow-up evaluation. As illustrated by FIG. 6, the SNP amplicon based genomic scarring assay on FFPE samples was predictive as to patient response to Olaparib for the PSOC cohort. For example, FIG. 6 shows that patients in the PSOC cohort with a high HRRD score were more likely to experience complete or partial remission (CR or PR, respectively). For this study, a cut-off score of 30 may be used to classify HRRD-positive versus HRRD-negative samples. It is understood that this cut-off score may be selected by the user of the genomic scarring assay. In some cases, the cut-off may be based upon a prior empirical study of HRRD scores for cancer patients with known clinical outcomes. As such, the HRRD cut-off may be based in part upon the type of stage of a given cancer, the demographic of patients, or other factors.

### Example 4: Kits for Use with Genomic Scarring Assays According to the Disclosure

As noted above, the genomic scarring assays described herein may be used to analyze sequencing data generated using a single-plex MASTR-based test. To that end, the SureMASTR HRRD Scar kit was developed as a molecular assay for the semi-quantitative assessment of tumor genomic instability in genomic DNA isolated from formalin fixed, paraffin embedded (FFPE) or fresh frozen (FF) tumor tissue from primary or metastatic cancer. The SureMASTR HRRD Scar kit may be used in combination with a drMID for Illumina NGS systems kit to allow for Universal PCR-based incorporation of molecular identifiers (MIDs) or barcodes and the Next-Generation Sequencing (NGS) specific adapters of all amplicons generated using the SureMASTR HRRD Scar kit. The SureMASTR HRRD Scar kits and the drMID for Illumina NGS systems kit(s) serve as a frontend amplification test for sequence analysis on Illumina MiSeq or NextSeq. The technology is based on targeted resequencing and relies on Multiplex PCR amplification and NGS.

The SureMASTR HRRD Scar kit includes a PCR mix comprising specific oligonucleotide primers and dNTPs in a tricine buffer (pH 8.0) containing BSA, KCl and MgCl₂; Taq DNA polymerase, and Amplification Reagent 1. A user may use the kit to amplify genomic DNA extracted from an FFPE or FFT sample, or cfDNA (e.g., isolated from plasma), using a single-tube multiplex PCR. The resulting amplification product may then be sequenced and analyzed using the methods described herein to determine an HRRD score for the sample from which the genomic DNA was extracted, and/or to diagnose the human subject or to select a treatment for the human subject (e.g., to determine whether a PARP inhibitor is likely to be effective).

In some aspects, the methods described herein may be performed in whole or in part using a general-purpose computer system. For example, sequencing results obtained for a given sample may be analyzed by software which implements the ASCAT algorithm and/or other processing steps necessary to generate an HRRD score, as described herein.

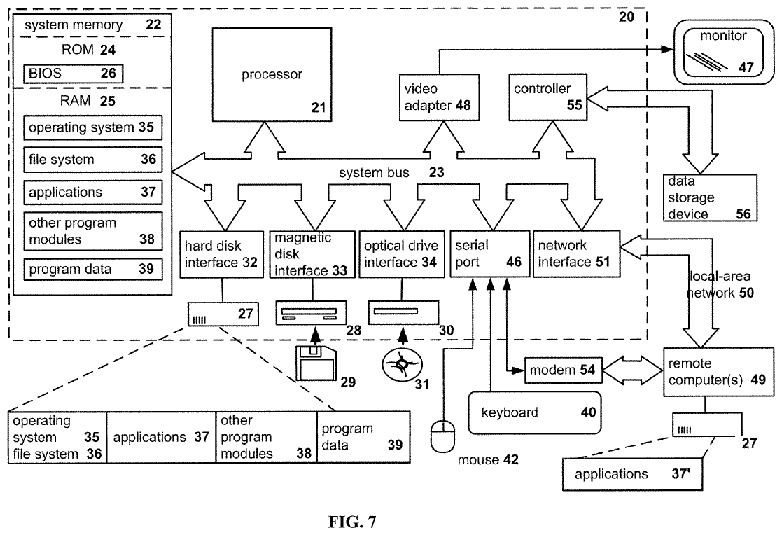

FIG. 7 illustrates an example of a general-purpose computer system (which may be a personal computer or a server) on which the disclosed systems and methods may be implemented. As shown, the computer system includes a central processing unit 21, a system memory 22 and a system bus 23 connecting the various system components, including the memory associated with the central processing unit M. The system bus 23 is realized like any bus structure known from the prior art, containing in turn a bus memory or bus memory controller, a peripheral bus and a local bus, which is able to interact with any other bus architecture. The system memory includes permanent memory (ROM) 24 and random-access memory (RAM) 25. The basic input/output system (BIOS) 26 includes the basic procedures ensuring the transfer of information between elements of the personal computer 20, such as those at the time of loading the operating system with the use of the ROM 24.

The personal computer 20, in turn, includes a hard disk 27 for reading and writing of data a magnetic disk drive 28 for reading and writing on removable magnetic disks 29 and an optical drive 30 for reading and writing on removable optical disks 31 such as CD-ROM, DVD-ROM and other optical information media. The hard disk 27, the magnetic disk drive 28, and the optical drive 30 are connected to the system bus 23 across the hard disk interface 32, the magnetic disk interface 33 and the optical drive interface 34, respectively. The drives and the corresponding computer information media are power-independent modules for storage of computer instructions, data structures, program modules and other data of the personal computer 20.

The present disclosure provides the implementation of a system that uses a hard disk 27, a removable magnetic disk 29 and a removable optical disk 31, but it should be understood that it is possible to employ other types of computer information media 56 which are able to store data in a form readable by a computer (solid state drives, flash memory cards, digital disks, random-access memory (RAM) and so on), which are connected to the system bus 23 via the controller 55.

The computer 20 has a file system 36, where the recorded operating system 35 is kept, and also additional program applications 37, other program modules 38 and program data 39, The user is able to enter commands and information into the personal computer 20 by using input devices (keyboard 40, mouse 42). Other input devices (not shown) can be used: microphone, joystick, game controller, scanner, and so on. Such input devices usually plug into the computer system 20 through a serial port 46, which in turn is connected to the system bus, but they can be connected in other ways, for example, with the aid of a parallel port, a game port or a universal serial bus (USW), A monitor 47 or other type of display device is also connected to the system bus 23 across an interface, such as a video adapter 48, in addition to the monitor 47, the personal computer can be equipped with other peripheral output devices (not shown), such as loudspeakers, a printer, and so on.

The personal computer 20 is able to operate in a network environment, using a network connection to one or more remote computers 49, The remote computer (or computers) 49 are also personal computers or servers having the majority or all of the aforementioned elements in describing the nature of a personal computer 20, as shown in FIG. 7. Other devices can also be present in the computer network, such as routers, network stations, peer devices or other network nodes.

Network connections can form a local-area computer network (LAN) 50 and a wide-area computer network (WAN). Such networks are used in corporate computer networks and internal company networks, and they generally have access to the Internet. In LAN or WAN networks, the personal computer 20 is connected to the local-area network 50 across a network adapter or network interface 51. When networks are used, the personal computer 20 can employ a modem 54 or other modules for providing communications with a wide-area computer network such as the Internet. The modem 54, which is an internal or external device, is connected to the system bus 23 by a serial port 46. It should be noted that the network connections are only examples and need not depict the exact configuration of the network, i.e., in reality there are other ways of establishing a connection of one computer to another by technical communication modules.

In various aspects, the systems and methods described herein may be implemented in hardware, software, firmware, or any combination thereof. If implemented in software, the methods may be stored as one or more instructions or code on a non-transitory computer-readable medium. Computer-readable medium includes data storage. By way of example, and not limitation, such computer-readable medium can comprise RAM, ROM, EEPROM, CD-ROM, Flash memory or other types of electric, magnetic, or optical storage medium, or any other medium that can be used to carry or store desired program code in the form of instructions or data structures and that can be accessed by a processor of a general purpose computer.

All statements herein reciting principles, aspects, and embodiments of the disclosure as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present disclosure, therefore, is not intended to be limited to the exemplary embodiments shown and described herein. Rather, the scope and spirit of present disclosure is embodied by the appended claims.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

1. A method for
predicting homologous recombination repair deficiency (HRRD), comprising the steps of:
a) providing a biological specimen obtained from a human subject, wherein the specimen comprises genomic DNA;
b) performing a multiplex polymerase chain reaction (PCR) assay on the genomic DNA to generate an amplified product, wherein the PCR assay is configured to amplify a plurality of amplicons;
c) sequencing at least a portion of the amplified product to generate sequencing results; and
d) determining a set of parameters of the biological specimen based on the sequencing results, wherein the set of parameters comprises:
i) a segment size parameter,
ii) a breakpoint count per unit-length parameter, and
iii) a copy number parameter
or
predicting homologous recombination repair deficiency (HRRD), comprising the steps of:
a) performing a multiplex polymerase chain reaction (PCR) assay on genomic DNA obtained from a human subject, to generate an amplified product comprising a plurality of amplicons which contain single-nucleotide polymorphisms (SNPs);
b) determining beta-allele frequency (BAF) and copy number parameters for each of the SNPs;
c) identifying a plurality of genomic segments, based on the BAF and copy number parameters for each of the SNPs, using an ASCAT algorithm;
d) determining the posterior probabilities for three components of a mixture model, using the genomic segments, wherein the components comprise a segment size, a breakpoint count per unit-length, and a copy number; and
e) calculating an HRRD score using a linear model, based on the posterior probabilities for the three components of the mixture model
or
predicting homologous recombination repair deficiency (HRRD), comprising:
a) providing an electronic device comprising one or more processors:
b) receiving, by the electronic device, sequencing results for an amplification product generated by a multiplex PCR using genomic DNA obtained from a human subject, wherein the sequencing results comprise sequences for a plurality of amplicons which contain SNPs;
c) determining, by the electronic device, beta-allele frequency (BAF) and copy number parameters for each of the SNPs;
d) identifying, by the electronic device, a plurality of genomic segments, based on the BAF and copy number parameters for each of the SNPs, using an ASCAT algorithm;
e) determining, by the electronic device, the posterior probabilities for three components of a mixture model, based on the genomic segments, wherein the components comprise a segment size, a breakpoint count per unit-length, and a copy number; and
f) calculating, by the electronic device, an HRRD score using a linear model, based on the posterior probabilities for the three components of the mixture model
or
amplifying genomic DNA, comprising:
a) obtaining genomic DNA from a specimen obtained from a human subject known to or suspected of having a cancer; and
b) amplifying a plurality of amplicons which contain single-nucleotide polymorphisms (SNPs) by performing a multiplex PCR using the genomic DNA;
wherein the multiplex PCR is performed using a set of PCR primers configured to amplify at least 5,000 amplicons spanning across all 22 human somatic chromosomes, wherein each amplicon comprises a SNP
or
generating a PCR amplification product, comprising:
a) obtaining genomic DNA from a specimen obtained from a human subject known to or suspected of having a cancer; and
b) generating the PCR amplification product by amplifying a plurality of amplicons which each contain a single-nucleotide polymorphisms (SNP) by performing a multiplex PCR using the genomic DNA:
wherein the multiplex PCR is performed using a set of PCR primers configured to amplify at least 5,000 amplicons spanning across all 22 human somatic chromosomes.

2. The method of claim 1, further comprising:
step e) predicting whether the biological specimen was obtained from a cell, tissue, or tumor that has an HRRD based upon the determined set of parameters.

3. The method of claim 1, further comprising step e) selecting a treatment for the human subject from which the biological specimen was obtained based upon the determined set of parameters.

4. The method of claim 1, further comprising step e) predicting the human subject's response to a cancer treatment regimen comprising a DNA damaging agent, an anthracycline, a topoisomerase I inhibitor, radiation, and/or a poly ADP-ribose polymerase (PARP) inhibitor, based upon the determined set of parameters.

5. The method of claim 2, wherein the segment size parameter, the breakpoint count per unit-length parameter, and the copy number parameter are combined to generate an aggregate score, prior to step e).

6. The method of claim 1, wherein the segment size parameter is determined by:
identifying a plurality of segments, wherein a segment is defined as a part of the genomic DNA consisting of at least 3 consecutive amplicons containing a heterozygous polymorphic position, which have the same copy number;
determining a segment size distribution for the identified plurality of segments; and
calculating the posterior probability of a mixture component describing the segment size which was determined by mixture modeling on a development set.

7. The method of claim 1, wherein the plurality of segments comprises segments which each have:
a) a size within the range of 5-50 megabase pairs (MBp);
b) a size within the range of 1-10, 10-20, 20-30, 30-40, or 40-50 MBp in length;
c) a size of at least 5, 10, 15, 20, 25, 30, 35, 40, 45, or 50 MBp in length; or
d) a size of less than 5, 10, 15, 20, 25, 30, 35, 40, 45, or 50 MBp in length.

8. The method of claim 1, wherein the breakpoint count per unit-length parameter is determined by calculating the posterior probability of a mixture component describing the number of breakpoints which was determined by mixture modeling on a development set.

9. The method of claim 8, wherein breakpoint count per unit-length is calculated for a portion of the genomic DNA, wherein the portion consists of one or more chromosomes or chromosome arms present within the genomic DNA.

10. The method of claim 1, wherein the copy number parameter is
determined by calculating the number of copies of one or more segments of the genomic DNA, wherein a segment is defined as a part of the genomic DNA consisting of at least 3 consecutive amplicons containing a heterozygous polymorphic position, which have the same copy number.

11. The method of claim 10, wherein the copy number parameter is calculated based on a plurality of segments of the genomic DNA.

12. The method of claim 11, wherein the copy number parameter is calculated based on at least 5, 10, 15, 20, 25, 30, 35, 40, 45, 50, 100, 150, 200, 250, or 500 segments.

13. The method of claim 11, wherein the copy number parameter is:
a) based on a plurality of segments of the genomic DNA, and calculated by determining the posterior probability of a mixture component describing the copy number of the plurality of segments, which was determined by mixture modeling on a development set; and/or
b) based at least in part on a categorization of the plurality of segments based upon their respective ploidy values.

14. The method of claim 1, wherein the biological specimen was obtained a) from a healthy human subject or b) from a human subject that has been diagnosed with or is suspected of having a cancer.

15. The method of claim 1, wherein the genomic DNA comprises a euploid genome and the PCR assay is configured to amplify at least 1,000; 2,000; 3,000; 4,000; 5,000; 6,000; 7,000; or 8,000 amplicons.

16. The method of claim 1, wherein each amplicon contains at least one polymorphic position having an average population frequency of a minor allele of at least 10, 11, 12, 13, 14, 15, 16, 17, 18, 19, 20, 21, 22, 23, 24, or 25%.

17. (canceled)

18. The method of claim 64, wherein the cancer treatment is a DNA damaging agent, an anthracycline, a topoisomerase I inhibitor, radiation, and/or a poly ADP-ribose polymerase (PARP) inhibitor.

19. (canceled)

20. The method of claim 1, further comprising
step f) predicting whether the genomic DNA was obtained from a cell, tissue, or tumor that has an HRRD based upon the HRRD score or
step f) predicting the human subject's response to a cancer treatment regimen comprising a DNA damaging agent, an anthracycline, a topoisomerase I inhibitor, radiation, and/or a poly ADP-ribose polymerase (PARP) inhibitor, based upon the HRRD score.

21-48. (canceled)

49. A system for predicting homologous recombination repair deficiency (HRRD), comprising:
an electronic device comprising one or more processors, configured to
receive sequencing results for an amplification product generated by a multiplex PCR using genomic DNA obtained from a human subject, wherein the sequencing results comprise sequences for a plurality of amplicons which contain SNPs;
determine beta-allele frequency (BAF) and copy number parameters for each of the SNPs;
identify a plurality of genomic segments, based on the BAF and copy number parameters for each of the SNPs, using an ASCAT algorithm;
determine the posterior probabilities for three components of a mixture model, based on the genomic segments, wherein the components comprise a segment size, a breakpoint count per unit-length, and a copy number; and
calculate an HRRD score using a linear model, based on the posterior probabilities for the three components of the mixture model
or
a system for predicting homologous recombination repair deficiency (HRRD), comprising: an electronic device comprising one or more processors, configured to perform one or more steps of claim 1.

50-63. (canceled)

64. A method of treating a cancer, comprising:
administering a cancer treatment to a human subject who has been diagnosed with an HRRD based on the methods described in claim 1,
or
a) receiving, by an electronic device, sequencing results for an amplification product generated by a multiplex PCR using genomic DNA obtained from a tumor found in a human subject, wherein the sequencing results comprise sequences for a plurality of amplicons which contain SNPs;
b) determining, by the electronic device, beta-allele frequency (BAF) and copy number parameters for each of the SNPs;
c) identifying, by the electronic device, a plurality of genomic segments, based on the BAF and copy number parameters for each of the SNPs, using an ASCAT algorithm;
d) determining, by the electronic device, the posterior probabilities for three components of a mixture model, based on the genomic segments, wherein the components comprise a segment size, a breakpoint count per unit-length, and a copy number;
e) calculating, by the electronic device, an HRRD score using a linear model, based on the posterior probabilities for the three components of the mixture model; and
f) selecting and/or administering a cancer treatment for the subject based on the HRRD score,
or
a) obtaining genomic DNA from a specimen obtained from a human subject known to or suspected of having a cancer; and
b) amplifying a plurality of amplicons which contain single-nucleotide polymorphisms (SNPs) by performing a multiplex PCR using the genomic DNA, wherein the multiplex PCR is performed using a set of PCR primers configured to amplify at least 5,000 amplicons spanning across all 22 human somatic chromosomes, wherein each amplicon comprises a SNP;
c) determining a homologous recombination repair deficiency (HRRD) score for the human subject, based on the sequences of the plurality of amplicons; and
d) selecting and/or administering a cancer treatment for the subject based on the HRRD score.

65. (canceled)

66. (canceled)

67. (canceled)

68. (canceled)

69. (canceled)

70. (canceled)
